(19) United States
(12) Patent Application Publication
(10) Pub. No.: US 2011/0052722 A1
(43) Pub. Date: Mar. 3, 2011

(54) Toleration Iron Supplement Compositions

(75) Inventors: Peter J. Krebs, Tucker, GA (US); Allison Krebs-Bensch, Decatur, GA (US); Jerome W. Mincy, Seymour, IN (US)

(73) Assignee: Grace Procurements LLC, Decatur, GA (US)

(21) Appl. No.: 12/941,571

(22) Filed: Nov. 8, 2010

Related U.S. Application Data
(63) Continuation of application No. 11/243,043, filed on Oct. 4, 2005, now abandoned.

(51) Int. Cl. A61K 33/26; A61K 31/715; A61P 3/02

(52) U.S. Cl. 424/648; 514/54

(57) ABSTRACT

The invention relates to compositions and methods for the treatment or prophylaxis of iron deficiency, and in particular of iron deficiency anemia, by administering a composition containing an effective amount of a pharmaceutically acceptable ferrous iron salt; and an effective amount of polysaccharide iron complex.

### BACKGROUND OF THE INVENTION

1. Field of the Invention

The present invention relates to compositions for treating or preventing iron deficiency in mammals, and in particular in humans, by administering to iron deficient or potentially iron deficient mammals an effective amount of a composition containing a pharmaceutically acceptable iron salt, and a polysaccharide iron complex.

2. Description of Related Art

Iron deficiency anemias are the most common form of anemia, and are perhaps the most common nutritional deficiency in the world. Iron deficiency anemias are associated with insufficient iron in the diet, poor absorption of iron by the body, and/or loss of blood. These conditions can often arise in humans and other animals as the result of, or in connection with physiological events, such as growth, pregnancy, menstruation, or in connection with pathological events, such as hemorrhage, food deficiencies. Iron deficiency anemia affects about 20% of women, about 50% of pregnant women, and about 3% of men.

Because anemia develops as iron stored in the body is depleted, and because women generally have smaller stores of iron than men (and increased loss of iron through menstruation), women are at higher risk of suffering the effects of anemia than are men. However, men and post-menopausal women are also at risk of anemia if they suffer from gastrointestinal blood loss resulting from ulcers or certain types of cancer, or from the use of nonsteroidal anti-inflammatory drugs (NSAIDS). In addition, patients undergoing erythropoietin (EPO) therapy (e.g., those with various types of kidney disease) can also suffer iron deficiency anemia when their iron stores become depleted due to the increased use of iron in making new red blood cells. Groups at increased risk of anemia thus include pre-menopausal women, pregnant or lactating women (because of an increased need for iron), infants, children, or adolescents experiencing rapid growth (and thus an increased need for iron), women and men with poor dietary intake of iron, individuals with diseases or conditions resulting in gastrointestinal blood loss, and individuals with Gaucher disease.

Without sufficient iron in the blood, red blood cells cannot carry oxygen, necessary for the normal functioning of cells, efficiently through the body. Common symptoms of anemia include one or more of the following: pallor, fatigue, irritability, weakness, shortness of breath, sore tongue, brittle nails, pica (unusual food cravings), decreased appetite, headache, and blue tinged sclerae. Diagnosis of iron deficiency anemia is typically confirmed by low hematocrit and hemoglobin, small red blood cells, low serum ferritin, low transferring saturation levels, low serum iron levels, high iron binding capacity, and bloody stool.

Treatment of anemia, in addition to identifying the source of the iron deficiency, typically involves administering iron supplements, often with the co-administration of vitamin C to aid absorption of iron. While this can be done via intravenous or intramuscular injection where necessary, a highly tolerated oral dosage form is much more desirable. Ionic iron (ferrous) oral dosage forms, such as ferrous sulfate, ferrous gluconate, ferrous succinate, and ferrous fumarate, are often used because the ferrous form of iron has better absorption than the less soluble ferric form, which can precipitate out in the body. Schmitt, J. of Renal Nutrition, 2, 126-128 (1992). These ferrous supplements are most efficiently absorbed on an empty stomach. However, this is not tolerable for many people due to gastrointestinal side effects; some individuals can take the oral dosage form with food. However, certain types of food (e.g., milk products and other foods high in calcium) can reduce the iron absorption efficiency, and for some patients, the gastrointestinal side effects limit the amount of iron supplement that can be prescribed. In addition, vitamin C administration may not be desirable for some patients (in particular those undergoing dialysis).

An alternative to ionic oral dosage forms of iron is polysaccharide iron complex (PIC), which has been shown to have equivalent efficacy at increasing hemoglobin and hematocrit values as ferrous sulfate and/or ferrous fumarate, but is better tolerated. Newton et al., Clinical Trials Journal, 17, 106-111 (1980); Piccinni et al., Pan. Med., 24, 213-220 (1982). PIC is a synthetic complex of ferric iron and carbohydrate. It does not suffer from some of the disadvantages of ferrous iron supplements in that it does not readily ionize and combine with inhibiting substances in the gut, while it remains soluble enough to cross the mucosal barrier of the intestinal lumen. PIC is believed to be absorbed via an active transport mechanism wherein iron is transferred at the surface of the intestinal mucosa to a carrier (transferring) for transport into the blood stream. PIC also seems to produce fewer gastrointestinal side effects than ferrous sulfate. Johnson et al., “A Prospective Open-Label Study Evaluating the Efficacy and Adverse Reactions of the use of Niferex®-150 in ESRD Patients Receiving EPOGEN®.” However, PIC is comparatively expensive compared to ferrous iron supplements.

As a result, while both ferrous salts and PIC are known as oral iron supplements that provide efficacy in treating iron deficiency anemia, they have been considered to be alternative treatments: if patients have difficulty with gastrointestinal side effects using the less expensive ferrous salts, then this regimen can be replaced with a more expensive, but better tolerated, PIC regimen to achieve equivalent efficacy. If patients lack the transferring necessary to effect PIC absorption, then a ferrous salt regimen can be used.

### SUMMARY OF INVENTION

The inventors have found that administering an oral iron supplement composition containing both ferrous iron salts and PIC provides an unexpectedly well tolerated method for treating iron deficiency anemia, and provides a composition that can be administered to a wide variety of patients, without regard to their ability to absorb iron through a particular physiological mechanism. In particular, the compositions of the invention provide therapeutic blood iron levels, with unexpectedly increased tolerability, irrespective of the patient's ability to absorb iron via a particular absorption mechanism. If the patient is unable to absorb ferrous iron (e.g., because of low ascorbic acid levels, gastrointestinal side effects, etc.), then sufficient iron is available through the PIC administered in the composition of the invention. If the patient is unable to absorb PIC because of insufficient transferrin in the lower gut, then sufficient iron is available through the ferrous iron administered.

As a result of this discovery, the present invention relates to an orally administrable iron supplement composition for treatment or prophylaxis of iron deficiency, comprising:

an effective amount of a pharmaceutically acceptable ferrous iron salt; and

an effective amount of polysaccharide iron complex.

The invention also relates to a method for the treatment or prophylaxis of iron deficiency, comprising:

administering to a patient in need thereof an effective amount of a composition comprising:

an effective amount of a pharmaceutically acceptable ferrous iron salt; and

an effective amount of polysaccharide iron complex.

### DETAILED DESCRIPTION OF SPECIFIC EMBODIMENTS

As described above, the invention relates to an effective amount of a pharmaceutically acceptable ferrous iron salt; and an effective amount of polysaccharide iron complex. The composition can also contain an effective amount of iron absorption enhancer, such ascorbic acid, and a pharmaceutically acceptable carrier, binder, or excipient, such as microcrystalline cellulose, sodium starch glycolate, magnesium stearate, alcohol, water or derivatives thereof.

The ferrous iron salt can be any ferrous iron salt conventionally used for treating iron deficiency anemia, but is desirably selected from the group consisting of ferrous fumarate, ferrous gluconate, ferrous sulfate, and ferrous succinate. Ferrous fumarate has been found to give particularly suitable results. The ferrous iron salt is typically present in amounts ranging between about 30 wt % and about 32 wt %, more particularly between about 20 wt % and about 25 wt %, based on the total weight of the composition.

The polysaccharide iron complex (PIC) may include any suitable PIC compound suitable for use as an iron supplement, such as that sold under the name Niferex®, FerUs 150, Iferex 150, Ferrex 150, Myferon 150 and PolyIron 150. The PIC is generally present in amounts ranging between about 38 wt % and about 46 wt % iron in the PIC and, more particularly between about 80 wt % and about 85 wt % in the formula.

If an iron enhancer is present, it is desirably included in an amount ranging between about 5 wt % and about 10 wt %, more particularly between about 5 wt % and about 10 wt %, based on the total weight of composition. As an alternative, an iron enhancer may be administered separately from the ferrous iron—PIC composition.

If present, the binder, carrier, or excipient is present in an amount ranging between about 5 wt % and about 10 wt %, more particularly between about 5 wt % and about 10 wt %, based on the total weight of composition.

In order to show the unexpectedly beneficial results obtained by the use of both ferrous iron and PIC, and to further explain the compositions and methods of the invention, the following experiments were carried out at the direction of the inventors.

### EXAMPLE

In order to compare the iron absorption and tolerability characteristics of ferrous fumarate, administered as a 487 mg/mL composition (comparative composition) and a composition containing ferrous fumarate and PIC, administered as a 348 mg/mL composition (inventive composition), Sprague Dawley Crl:CD(SD) rats were randomly assigned to 3 groups as indicated in the Table below:

TABLE

| | | | | Dose | | Dose |
|---|---|---|---|---|---|---|
| | | | | Level | Dose | Concen. |
| Group | Number of Animals | | | (mg | Volume | (mg |
| Number | Males | Females | Test Material | Fe/kg) | (mL/kg) | Fe/mL) |
| 1 | 10 | 10 | RO Water | 0 | 7.7 | 0 |
| 2 | 10 | 10 | Ferrous | 500 | 6.42 | 77.92 |
| | | | Fumarate | | | |
| 3 | 10 | 10 | Ferrous | 250 | 3.2 | 77.92 |
| | | | Fumarate | | | |
| | | | PIC | 250 | 4.5 | 55.68 |

The test material solution or vehicle control was administered once per day by oral gavage administration, and individual dose volumes were calculated based on the most recent body weight data for each animal.

General health/mortality and moribundity checks were performed twice daily, and detailed clinical observations were performed on days 1, 8, and 15 prior to dosing. Hematology parameters were evaluated for all animals on days 1, 7, and 14. Blood collection for serum iron concentration evaluation was performed on these days as well, and blood samples were obtained via the orbital plexus while the animals were under light isoflurane anesthesia. All animals were subjected to complete gross necropsy examination at scheduled euthanasia.

Blood samples for hematology testing were collected in volumes of about 200 μL in tubes containing anticoagulant K₂EDTA, and were evaluated for red blood cell count (RBC), hemoglobin concentration (Hb), hematocrit (Hct), mean corpuscular volume (MCV), mean corpuscular hemoglobin concentration (MCHC), mean corpuscular hemoglobin (MCH), reticulocyte count (Retic), red blood cell morphology, white blood cell count (WBC), neutrophil count (Neut), lymphocyte count (Lymph), monocyte count (Mono), eosinophil count (Eos), and basophil count (Baso). Serum iron was measured by taking about 1 mL blood samples in a serum separator tube.

Segmented neutrophils, platelets, and lymphocytes changed in similar manner for both Groups 2 and 3, indicating that the immune response to ferrous fumarate and to the inventive composition was about the same. Both Groups 2 and 3 exhibited similar significant increases in serum iron levels as well. However, Group 2 exhibited gross internal changes different from those exhibited by Group 3, namely body fat depletion, thyroid nodules, reddened mandibular lymph nodes, reddened stomach mucosa, small thymus, foci on the thymus, and dark fecal matter. By contrast, the only gross internal findings exhibited by Group 3 were a reddened thymus and dark fecal matter. Based on these results, it is concluded that both compositions provide equivalent efficacy at increasing serum iron levels, but that the Group 3 material (i.e., the composition of the invention) is significantly better tolerated than is ferrous fumarate alone, even though the dose concentration of ferrous fumarate is the same in both compositions. In other words, the results support the conclusion that the addition of PIC to ferrous fumarate surprisingly allows the same concentration of ferrous fumarate to be better tolerated than if the PIC was not added.

Without wishing to be bound by any theory, the increase in tolerability observed with the composition of the invention is believed to occur as the result of distributing the total iron content in the composition among compounds that provide iron to the patient's bloodstream via two different mechanisms. The ferrous iron salts are readily absorbed in the upper gut, by direct dissolution and absorption of the ferrous iron by the bloodstream. Iron available from PIC is absorbed in the lower gut via an active protein transport mechanism. Although the reason that this diversity of absorption mechanisms results in increased tolerability is not well understood, the result is believed to extend to any pharmaceutically acceptable ionic iron salt when administered in combination with PIC.

The composition of the present invention can desirably be administered in amounts ranging from about 4.0 mg/kg of ferrous salt (e.g., as ferrous fumarate) and 4.0 mg/kg of PIC, more particularly, from about 10.0 mg/kg of ferrous salt and 10.0 mg/kg of PIC, in order to obtain good efficacy at raising serum iron levels, while avoiding adverse reactions. The composition is typically administered as a liquid or elixir, combined with alcohol and water as an excipient, at a concentration ranging from about 200 mg/ml to about 600 mg/ml of ferrous fumarate and from about 200 mg/ml to about 600 mg/ml of PIC. Those of skill in the art will understand that different concentrations of each component can be administered, depending upon the degree of iron deficiency. However, the total administration should not exceed the toxicity limits of ferrous fumarate (about 200-250 mg/kg) or of PIC (about 5000 mg/kg).

1. An orally administrable iron supplement composition for treatment or prophylaxis of iron deficiency, comprising:
an effective amount of a pharmaceutically acceptable ferrous iron salt; and
an effective amount of polysaccharide iron complex, wherein the polysaccharide iron complex comprises from about 38 wt % to about 46 wt % iron.

2. The composition of claim 1, wherein the amount of iron derived from the pharmaceutically acceptable ferrous iron salt is the same as the amount of iron derived from the polysaccharide iron complex.

3. (canceled)

4. The composition of claim 1, further comprising a pharmaceutically acceptable binder, excipient, or carrier.

5. The composition of claim 1, further comprising an effective amount of an iron absorption enhancer.

6. The composition of claim 5, wherein the iron absorption enhancer is ascorbic acid or pharmaceutically acceptable salt thereof.

7. The composition of claim 5, wherein the iron absorption enhancer is present in an amount ranging between about 5 wt % and about 10 wt %, based on the total weight of the composition.

8. The composition of claim 1, wherein the ferrous iron salt is selected from the group consisting of ferrous sulfate, ferrous fumarate, ferrous succinate, and ferrous gluconate.

9. The composition of claim 8, wherein the ferrous iron salt is ferrous fumarate.

10. A method for the treatment or prophylaxis of iron deficiency, comprising:
administering to a patient in need thereof an effective amount of a composition comprising:
an effective amount of a pharmaceutically acceptable ferrous iron salt; and
an effective amount of polysaccharide iron complex.

11. The method of claim 10, wherein the amount of iron derived from pharmaceutically acceptable ferrous iron salt is the same as the amount of iron derived from polysaccharide iron complex.

12. The method of claim 10, wherein the polysaccharide iron complex is present in the composition in an amount ranging from about 38 wt % to about 46 wt %, based on the total weight of the composition.

13. The method of claim 10, wherein the composition further comprises a pharmaceutically acceptable binder, excipient, or carrier.

14. The method of claim 10, further comprising administering an effective amount of an iron absorption enhancer.

15. The method of claim 14, wherein the iron absorption enhancer is ascorbic acid or a pharmaceutically acceptable salt thereof.

16. The method of claim 10, wherein the ferrous iron salt is selected from the group consisting of ferrous sulfate, ferrous fumarate, ferrous succinate, and ferrous gluconate.

17. The method of claim 10, wherein the ferrous iron salt is ferrous fumarate.
